(19) United States
(12) Patent Application Publication
(10) Pub. No.: US 2005/0033050 A1
(43) Pub. Date: Feb. 10, 2005

(54) Phthalazinones and the use thereof in order to combat undesirable microorganisms

(76) Inventors: Bernd-Wieland Kruger, Gladbach (DE); Astrid Ullmann, Koln (DE); Stefan Hillebrand, Neuss (DE); Astrid Mauler-Machnik, Leichlingen (DE); Ulrike Wachendorff-Nuemann, Neuwied (DE)

Correspondence Address: BAYER CROPSCIENCE LP;Patent Department, 100 BAYER ROAD, PITTSBURGH, PA, 15205-9741, US

(21) Appl. No.: 10/489,085

(22) PCT Filed: Sep. 4, 2002
(86) PCT No.: PCT/EP02/09871

(30) Foreign Application Priority Data
Sep. 17, 2001 (DE) 101-45-771.5

(51) Int. Cl. C07D 237/30

(52) U.S. Cl. 544/237

(57) ABSTRACT

The invention relates to compounds of the formula (I), 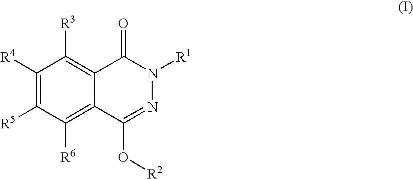
in which R¹, R², R³, R⁴, R⁵ and R⁶ are as defined in the disclosure, to a plurality of processes for their preparation, and to their use for controlling unwanted microorganisms.

The present invention relates to novel phthalazinones, to a plurality of processes for their preparation and to their use for controlling unwanted microorganisms.

It is already known that certain phthalazinones have fungicidal properties (compare, for example, JP-A-08 198 856). The activity of these compounds is good; however, at low application rates it is sometimes unsatisfactory.

This invention now provides novel phthalazinones of the formula (I), 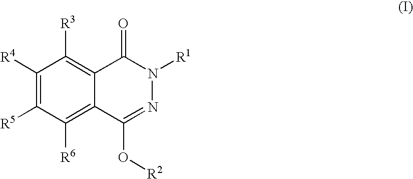
in which
R¹ and R² are identical or different and independently of one another represent alkyl having 2 to 12 carbon atoms, alkenyl, alkynyl, cycloalkyl, cycloalkylalkyl or alkoxyalkyl, and
R³, R⁴, R⁵ and R⁶ are identical or different and independently of one another represent hydrogen, halogen, cyano, nitro, alkyl, alkoxy, alkylthio, alkylsulphinyl, alkylsulphonyl, alkenyl, alkenyloxy, haloalkyl, haloalkoxy, haloalkylthio, haloalkylsulphinyl, haloalkylsulphonyl, haloalkenyl or haloalkenyloxy, hydroximinoalkyl, alkoximinoalkyl or cycloalkyl,
where at least one of the radicals R³, R⁴, R⁵ and R⁶ is different from hydrogen.

Furthermore, it has been found that phthalazinones of the formula (I) are obtained when
a) phthalazinediones of the formula (II), 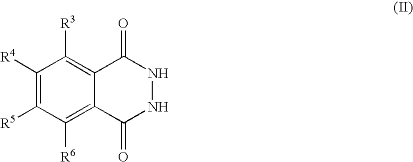
   in which
R³, R⁴, R⁵ and R⁶ are as defined above, are reacted with an alkyl derivative of the formula (III)
   R—X (III)
   in which
R has the meanings given above for R¹ and R² and
X represents a leaving group, preferably halogen, alkylsulphonyl or arylsulphonyl,
if appropriate in the presence of an acid acceptor and if appropriate in the presence of a diluent, or when
b) alkylphthalazinones of the formula (IV), 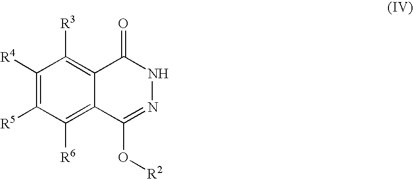
   in which
R², R³, R⁴, R⁵ and R⁶ have the meanings given above and below are reacted with an alkyl derivative of the formula (III),
   R—X (III)
   in which
R has the meaning given above for R¹ and
X is as defined above,
if appropriate in the presence of an acid acceptor and if appropriate in the presence of a diluent, or when
c) hydroxyphthalazinones of the formula (V) 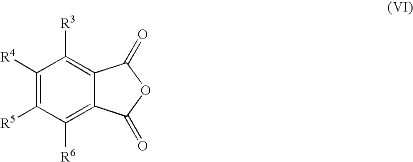
   are reacted with an alkyl derivative of the formula (III).
   R—X (III)
   in which
R has the meaning given above for R² and
X is as defined above,
if appropriate in the presence of an acid acceptor and if appropriate in the presence of a diluent

The compounds according to the invention can, if appropriate, be present as mixtures of different possible isomeric forms, in particular of stereoisomers, such as, for example, E and Z, threo and erythro, and also optical isomers, and, if appropriate, also of tautomers or regioisomers. What is claimed are both the E and the Z isomers, and the threo and erythro and also the optical isomers, possible regioisomers, any mixtures of these isomers, and the possible tautomeric forms.

Finally, it s been found that the novel phthalazinones of the formula (I) have very good microbiocidal properties and can be used for controlling unwanted microorganisms both in crop protection and in the protection of materials.

Surprisingly, the phthalazinones of the formula (I) according to the invention have considerably better fungicidal activity than the constitutionally most similar prior-art active compounds of the same direction of action.

Particular meanings of the substituents or ranges of the radicals listed in the formulae given above and below are illustrated below:
R¹ and R² are identical or different and independently of one another preferably represent alkyl, alkenyl or alkynyl having in each case 2 to 12 carbon atoms, cycloalkyl having 3 to 8 carbon atoms, cycloalkylalkyl having 3 to 8 carbon atoms in the cycloalkyl moiety and 1 to 6 carbon atoms in the alkyl moiety or alkoxyethyl, alkoxypropyl or alkoxybutyl having in each case 1 to 6 carbon atoms in the alkoxy moiety.
R³, R⁴, R⁵ and R⁶ are identical or different and independently of one another preferably represent hydrogen, halogen, cyano, nitro,
   in each case straight-chain or branched alkyl, alkoxy, alkylthio, alkylsulphinyl or alkylsulphonyl having in each case 1 to 6 carbon atoms;
   in each case straight-chain or branched alkenyl or alkenyloxy having in each case 2 to 6 carbon atoms;
   in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
   in each case straight-chain or branched haloalkenyl or haloalkenyloxy having in each case 2 to 6 carbon atoms and 1 to 11 identical or different halogen atoms;
   hydroxyiminoalkyl having 1 to 6 carbon atoms;
   alkoxyiminoalkyl having 2 to 6 carbon atoms or
   cycloalkyl having 3 to 6 carbon atoms,
   where at least one of the radicals R³, R⁴, R⁵ or R⁶ is different from hydrogen.
R¹ and R² are identical or different and independently of one another particularly preferably represent in each case straight-chain or branched ethyl,. propyl, butyl, pentyl, hexyl, heptyl, octyl, nonyl, decyl, undecyl or dodecyl; ethenyl, propenyl, butenyl, pentenyl, hexenyl, heptenyl, octenyl, nonenyl, decenyl, undecenyl or dodecenyl; ethynyl, propynyl, butynyl, pentynyl, hexynyl, heptynyl, octynyl, nonynyl, decynyl, undecynyl, dodecynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, cyclohexylethyl, methoxyethyl, ethoxyethyl, methoxypropyl or methoxybutyl, each of which may be attached in any position.
R³, R⁴, R⁵ and R⁶ are identical or different and independently of one another particularly preferably represent hydrogen, fluorine, chlorine, bromine, iodine, cyano, nitro, methyl, ethyl, n- or i-propyl, n-, i-, s- or t-butyl, methoxy, ethoxy, n- or i-propoxy, methylthio, ethylthio, n- or i-propylthio, methylsulphinyl, ethylsulphinyl, methylsulphonyl or ethylsulphonyl, trifluoromethyl, difluorochloromethyl, fluorodichloromethyl, trifluoroethyl, pentafluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl or trifluoromethylsulphonyl, methylsulphonyloxy, ethylsulphonyloxy, hydroximinomethyl, hydroximinoethyl, methoximinomethyl, ethoximinomethyl, methoximinoethyl or ethoximinoethyl, cyclopropyl, cyclobutyl, cyclopentyl or cyclohexyl,
where at least one of the radicals R³, R⁴, R⁵ or R⁶ is different from hydrogen.
R¹ very particularly preferably represents straight-chain or branched methyl, ethyl, propyl, butyl, ethenyl, propenyl, butenyl, ethynyl, propynyl, butynyl, cyclopropyl, cyclobutyl, cyclopentyl, cyclohexyl, cyclopropylmethyl, cyclobutylmethyl, cyclopentylmethyl, cyclohexylmethyl or methoxyethyl, each of which may be attached in any position.
R² very particularly preferably represents straight-chain or branched methyl, ethyl, propyl, butyl, pentyl, ethenyl, propenyl, butenyl, ethynyl, propynyl, butynyl, cyclopropyl, cyclobutyl, cyclopentyl, cyclohexyl, cyclopropylmethyl, cyclobutylmethyl, cyclopentylmethyl, cyclohexylmethyl or methoxyethyl, each of which may be attached in any position.
R³ very particularly preferably represents hydrogen.
R⁴ very particularly preferably represents hydrogen, n-propoxy, fluorine, chlorine, bromine or iodine.
R⁵ very particularly preferably represents hydrogen, n-propoxy, fluorine, chlorine, bromine or iodine.
R⁶ very particularly preferably represents hydrogen.

Saturated or unsaturated hydrocarbon radicals, such as alkyl or alkenyl, can in each case be straight-chain or branched, as far as this is possible, including in combination with heteroatoms, such as, for example, in alkoxy or hydroxyiminoalkyl. Unless indicated otherwise, preference is given to carbon chains of 1-6 carbon atoms.

Halogen-substituted radicals, such as, for example, haloalkyl, are mono- or polyhalogenated. In the case of polyhalogenation, the halogen atoms can be identical or different. Unless indicated otherwise, preference is given to carbon chains of 1-6 carbon atoms.

Halogen represents fluorine, chlorine, bromine and iodine, particularly preferably fluorine, chlorine and bromine.

Cycloalkyl represents saturated carbocyclic compounds which may form a polycyclic ring system with further carbocyclic fused-on or bridged rings. Unless indicated otherwise, preference is given to carbocycles having 3 to 6 carbon atoms.

However, the general, preferred or particularly preferred radical definitions or illustrations listed above can also be combined with one another as desired, i.e. between the respective ranges and preferred ranges. The definitions apply both to the end products and, correspondingly, to the precursors and intermediates. Moreover, individual definitions may also not apply.

The process a) according to the invention can be illustrated by the reaction equation below:

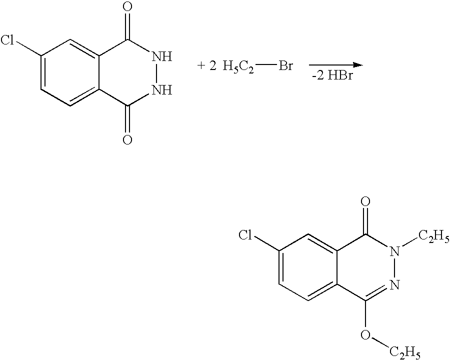

The formula (II) provides a general definition of the phthalazinediones required as starting materials for carrying out the process a) according to the invention. In this formula (II), R³, R⁴, R⁵ and R⁶ preferably or particularly preferably have those meanings which have already been mentioned in connection with the description of the compounds of the formula (I) according to the invention as being preferred, particularly preferred or very particularly preferred for R³, R⁴, R⁵ and R⁶. The phthalazinediones of the formula (II) are known and can be prepared by known methods (compare, for example, B. J. Chem. Soc., Perkin Trans. 1 (1980), (8), 1834-40).

The formula (III) provides a general definition of the alkyl derivatives furthermore required as starting materials for carrying out the process a) according to the invention. In this formula (III), R preferably or particularly preferably has that meaning which has already been given in connection with the description of the compounds of the formula (I) according to the invention as being preferred or as being particularly preferred for R¹ or R². X represents halogen, preferably bromine or iodine, or represents alkylsulphonyl, preferably methylsulphonyl, or represents arylsulphonyl, preferably 4-tolylsulphonyl. The alkyl derivatives of the formula (III) are known chemicals of synthesis.

The process b) according to the invention can be illustrated by the reaction equation below:

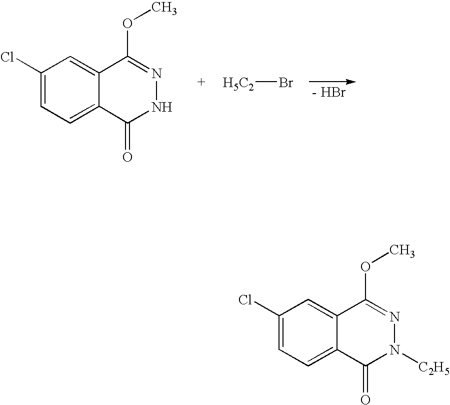

The formula (IV) provides a general definition of the alkylphthalazinones required as starting materials for carrying out the process b) according to the invention. In this formula (IV), R², R³, R⁴, R⁵ and R⁶ preferably have those meanings which have already been mentioned in connection with the description of the compounds of the formula (I) according to the invention as being preferred or as being particularly preferred for R², R³, R⁴, R⁵ and R⁶.

The alkylphthalazinones of the formula (IV) are novel and also form part of the subject-matter of the present application. Furthermore, it has been found that the novel alkylphthalazinones of the formula (IV), too, have very good microbicidal properties and can be used for controlling unwanted microorganisms both in crop protection and in the protection of materials.

They are obtained when (process d) phthalazinediones of the formula (II) are reacted with an alkyl derivative of the formula (III), if appropriate in the presence of an acid acceptor and if appropriate in the presence of a diluent.

The phthalazinediones of the formula (II) required as starting materials for carrying out the process d) according to the invention have already been described further above in connection with the description of the process a) according to the invention.

The alkyl derivatives of the formula (III) furthermore required as starting materials for carrying out the processes b) and d) according to the invention have already been described further above in connection with the description of the process a) according to the invention.

The process c) according to the invention can be illustrated by the reaction equation below:

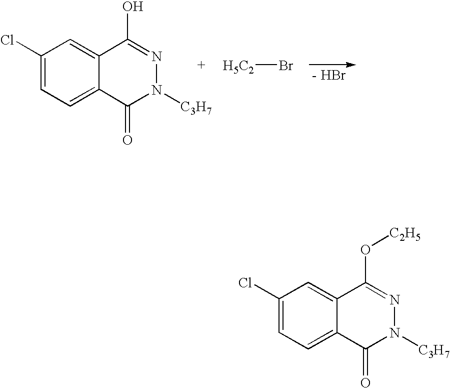

The alkyl derivatives of the formula (III) furthermore required as starting materials for carrying out the process c) according to the invention have already been described further above in connection with the description of the process a) according to the invention.

The formula (V) provides a general definition of the hydroxyphthalazinones furthermore required as starting materials for carrying out the process c) according to the invention. In this formula (V), R¹, R³, R⁴, R⁵ and R⁶ preferably have those meanings which have already been mentioned in connection with the description of the compounds of the formula (I) according to the invention as being preferred or as being particularly preferred for R¹, R³, R⁴, R⁵ and R⁶.

The hydroxyphthalazinones of the formula (V) are novel and also form part of the subject-matter of the present application. Furthermore, it has been found that the novel hydroxyphthalazinones of the formula (V), too, have very good microbicidal properties and can be used for controlling unwanted microorganisms both in crop protection and in the protection of materials.

They are obtained when (process e) phthalic anhydrides of the formula (VI) 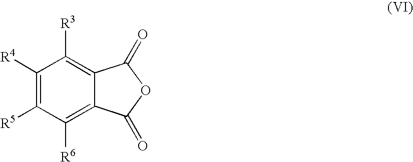
in which
R³, R⁴, R⁵ and R⁶ are as defined above, are reacted with a hydrazine derivative of the formula (VII)
   H₂N—NH—R¹ (VII)
   in which
R¹ is as defined above,
or a salt thereof,
if appropriate in the presence of a diluent and if appropriate in the presence of a salt.

When the hydroxyphthalazinones of the formula (V) are prepared according to process e), in many cases mixtures of in each case two regioisomers are obtained. These mixtures can be used as starting materials for preparing the compounds of the formula (I) according to the process c), even without separation into the individual components.

The process e) according to the invention can be illustrated by the reaction equation below:

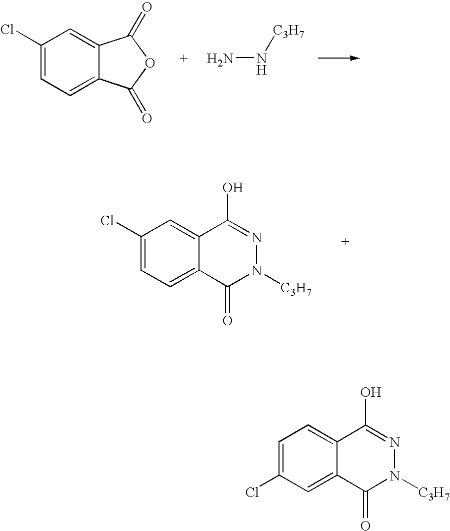

The formula (VI) provides a general definition of the phthalic anhydrides required as starting materials for carrying out the process e) according to the invention. In this formula (VI), R³, R⁴, R⁵ and R⁶ preferably have those meanings which have already been mentioned in connection with the description of the compounds of the formula (I) according to the invention as being preferred or as being particularly preferred for R³, R⁴, R⁵ and R⁶. The phthalic anhydrides of the formula (VI) are known and can be obtained by known methods (compare, for example, J. Chem. Soc., Perkin Trans. I 1980, 1834-1840).

The formula (VII) provides a general definition of the hydrazine derivatives furthermore required as starting materials for carrying out the process e) according to the invention. In this formula (VII), R¹ preferably has that meaning which has already been mentioned in connection with the description of the compounds of the formula (I) according to the invention as being preferred or as being particularly preferred for R¹. If salts of the hydrazine derivatives are used, preference is given to the hydrochlorides and the hydrogen sulphates. The hydrazine derivatives of the formula (VII) and their salts are known and can be obtained by known methods (compare, for example, J. Synth. Commun. 1995, 3805-3812).

Suitable diluents for carrying out the processes a), b), c), d) and e) according to the invention are water and all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl-t-butyl ether, methyl-t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethylsulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water.

The processes a), b), c) and d) according to the invention are, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropyl amide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethyl-benzylamine, pyridine, N-methyl-piperidine, N-methylmorpholine, N,N-dimethylaminopyridine, diazabicyclooctane (DABCO), diazabicyclononene (DBN) or diazabicycloundecene (DBU).

When carrying out the processes a), b), c) and d) according to the invention, the reaction temperatures can be varied within a relatively wide range. In general, the processes are carried out at temperatures of from −20° C. to 150° C., preferably at temperatures of from −10° C. to 80° C.

For carrying out the process a) according to the invention for preparing the compounds of the formula (I), in general from 2 to 15 mol, preferably from 2 to 5 mol, of alkyl derivative of the formula (III) are employed per mole of the phthalazinedione of the formula (II).

For carrying out the process b) according to the invention for preparing the compounds of the formula (I), in general from 1 to 10 mol, preferably from 1 to 5 mol, of alkyl derivative of the formula (III) are employed per mole of the alkylphthalazinone of the formula (IV).

For carrying out the process c) according to the invention for preparing the compounds of the formula (I), in general from 1 to 15 mol, preferably from 1 to 8 mol, of alkyl derivative of the formula (III) are employed per mole of the hydroxyphthalazinone of the formula (V).

For carrying out the process d) according to the invention for preparing the compounds of the formula (IV), in general from 1 to 2 mol, preferably from 1 to 5 mol, of alkyl derivative of the formula (III) are employed per mole of the phthalazinedione of the formula (II).

Suitable diluents for carrying out the process e) according to the invention are inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole, and also carboxylic acids, such as acetic acid.

The process e) according to the invention is, if appropriate, carried out in the presence of a salt. Suitable salts are, preferably, acetates, such as, for example, sodium acetate.

When carrying out the process e) according to the invention, the reaction temperatures can be varied within a relatively wide range. In general, the process is carried out at temperatures of from 0° C. to 200° C., preferably at temperatures of from 20° C. to 120° C.

For carrying out the process e) according to the invention for preparing the compounds of the formula (I), in general from 1 to 15 mol, preferably from 1 to 8 mol, of hydrazine derivative of the formula (VII) are employed per mole of the phthalic anhydride of the formula (VI).

All processes according to the invention are generally carried out under atmospheric pressure. However, it is also possible to operate under elevated or reduced pressure—in general between 0.1 bar and 10 bar.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or
Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and Pseudocercosporella species, such as, for example, Pseudocercosporella herpotrichoides.

The active compounds according to the invention also show a strong invigorating action in plants. Accordingly, they are suitable for mobilizing the internal defences of the plant against attack by unwanted microorganisms.

In the present context, plant-invigorating (resistance-inducing) compounds are to be understood as meaning substances which are capable of stimulating the defence system of plants such that, when the treated plants are subsequently inoculated with unwanted microorganisms, they develop substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time within which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be employed with particularly good results for controlling cereal diseases, such as, for example, against Erysiphe species, diseases in viticulture, fruit and vegetable cultivation, such as, for example, against Sphaerotheca species.

The active compounds according to the invention are also suitable for increasing the yield of crops. In addition, they show reduced toxicity and are well tolerated by plants. If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products which exceed the effects which were actually to be expected are possible.

The transgenic plants or plant cultivars (i.e. those obtained by genetic engineering) which are preferred and are to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized in particular are increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as. “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosates or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosates, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants.

Particular emphasis is given to the treatment of plants with the compounds or mixtures specifically mentioned in the present text.

The treatment of the plants and parts of plants according to the invention with the active compounds is carried out directly or by action on their environment, habitat or storage area according to customary treatment methods, for example by dipping, spraying, evaporating, atomizing, broadcasting, brushing on and, in the case of propagation material, in particular in the case of seeds, furthermore by one- or multi-layer coating.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably tackifiers, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes) and against slime organisms and algae.

Microorganisms of the following genera may be mentioned as examples:
Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and. microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 per cent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

aldimorph, ampropylfos, ampropylfos-potassium, andoprim, anilazine, azaconazole, azoxystrobin,
benalaxyl, benodanil, benomyl, benzamacril, benzamacryl-isobutyl, bialaphos, binapacryl, biphenyl, bitertanol, blasticidin-S, bromuconazole, bupirimate, buthiobat,
calcium polysulphide, carpropamid, capsimycin, captafol, captan, carbendazim, carboxin, carvone, chinomethionat (quinomethionat), chlobenthiazone, chlorfenazole, chloroneb, chloropicrin, chlorothalonil, chlozolinate, clozylacon, cufraneb, cymoxanil, cyproconazole, cyprodinil, cyprofuram,
debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,
ediphenphos, epoxiconazole, etaconazole, ethirimol, etridiazole,
famoxadone, fenapanil, fenarimol, fenbuconazole, fenfuram, fenhexamid, fenitropan, fenpiclonil, fenpropidin, fenpropimorph, fentin-acetate, fentin-hydroxide, ferbam, ferimzon, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,
guazatine,
hexachlorobenzene, hexaconazole, hymexazole,
imazalil, imibenconazole, iminoctadin, iminoctadine albesilat, iminoctadine triacetate, iodocarb, ipconazole, iprobenfos (IBP), iprodione, iprovalicarb, irumamycin, isoprothiolane, isovaledione,
kasugamycin, kresoxim-methyl, copper preparations such as copper hydroxide, copper naphthenate, copper oxychloride, copper sulphate, copper oxide, oxine copper and Bordeaux mixture,
mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,
nickel dimethyldithiocarbamate, nitrothal-isopropyl, nuarimol,
ofurace, oxadixyl, oxamocarb, oxolinic acid, oxycarboxim, oxyfenthiin,
paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,
quinconazole, quintozene (PCNB), quinoxyfen,
sulphur and sulphur preparations, spiroxamine,
tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazol, tridemorph, trifloxystrobin, triflumizole, triforine, triticonazole,
uniconazole,
validamycin A, vinclozolin, viniconazole,
zarilamid, zineb, ziram and also
Dagger G,
OK-8705,
OK-8801,
α-(1,1-dimethylethyl)-β-(2-phenoxyethyl)-1 H-1,2,4-triazole-1-ethanol,
α-(2,4-dichlorophenyl)-β-fluoro-β-propyl-1H-1,2,4-triazole-1-ethanol,
α-(2,4-dichlorophenyl)-β-methoxy-α-methyl-1H-1,2,4-triazole-1-ethanol,
α-(5-methyl-1,3-dioxan-5-yl)-β-[[4-(trifluoromethyl)-phenyl]-methylene]-1H-1,2,4-triazole-1-ethanol,
(5RS,6RS)-6-hydroxy-2,2,7,7-tetramethyl-5-(1H-1,2,4-triazol-1-yl)-3-octanone,
(E)-α-(methoxyimino)-N-methyl-2-phenoxy-phenylacetamide,
1-(2,4-dichlorophenyl)-2-(1H-1,2,4-triazol-1-yl)-ethanone-O-(phenylmethyl)-oxime,
1-(2-methyl-1-naphthalenyl)-1H-pyrrole-2,5-dione,
-(3,5-dichlorophenyl)-3-(2-propenyl)-2,5-pyrrolidinedione,
1-[(diiodomethyl)-sulphonyl]-4-methyl-benzene,
1-[[2-(2,4-dichlorophenyl)-1,3-dioxolan-2-yl]-methyl]-1H-imidazole,
1-[[2-(4-chlorophenyl)-3-phenyloxiranyl]-methyl]-1H-1,2,4-triazole,
1-[1-[2-[(2,4-dichlorophenyl)-methoxy]-phenyl]-ethenyl]-1H-imidazole,
1-methyl-5-nonyl-2-(phenylmethyl)-3-pyrrolidinole,
2′,6′-dibromo-2-methyl-4′-trifluoromethoxy-4′-trifluoro-methyl-1,3-thiazole-5-carboxanilide,
2,6-dichloro-5-(methylthio)-4-pyrimidinyl-thiocyanate,
2,6-dichloro-N-(4-trifluoromethylbenzyl)-benzamide,
2,6-dichloro-N-[[4-(trifluoromethyl)-phenyl]-methyl]-benzamide,
2-(2,3,3-triiodo-2-propenyl)-2H-tetrazole,
2-[(1-methylethyl)-sulphonyl]-5-(trichloromethyl)-1,3,4-thiadiazole,
2-[[6-deoxy4-O-(4-O-methyl-β-D-glycopyranosyl)-α-D-glucopyranosyl]-amino]-4-methoxy-1H-pyrrolo[2,3-d]pyrimidin-5-carbonitrile,
2-aminobutane,
2-bromo-2-(bromomethyl)-pentanedinitrile,
2-chloro-N-(2,3-dihydro-1,1,3-trimethyl-1H-inden-4-yl)-3-pyridinecarboxamide,
2-chloro-N-(2,6-dimethylphenyl)-N-(isothiocyanatomethyl)-acetamide,
2-phenylphenol (OPP),
3,4-dichloro-1-[4-(difluoromethoxy)-phenyl]-1H-pyrrole-2,5-dione,
3,5-dichloro-N-[cyano[(1-methyl-2-propynyl)-oxy]-methyl]-benzamide,
3-(1,1-dimethylpropyl)-1-oxo-1H-indene-2-carbonitrile,
3-[2-(4-chlorophenyl)-5-ethoxy-3-isoxazolidinyl]-pyridine,
4-chloro-2-cyano-N,N-dimethyl-5-(4-methylphenyl)-1H-imidazole-1-sulphonamide,
4-methyl-tetrazolo[1,5-a]quinazolin-5(4H)-one,
8-hydroxyquinoline sulphate,
9H-xanthene-2-[(phenylamino)-carbonyl]-9-carboxylic hydrazide,
bis-(1-methylethyl)-3-methyl-4-[(3-methylbenzoyl)-oxy]-2,5-thiophenedicarboxylate,
cis-1-(4-chlorophenyl)-2-(1H-1,2,4-triazol-1-yl)-cycloheptanol,
cis-4-[3-[4-(1,1-dimethylpropyl)-phenyl-2-methylpropyl]-2,6-dimethyl-morpholine hydrochloride,
ethyl[(4-chlorophenyl)-azo]-cyanoacetate,
potassium bicarbonate,
methanetetrathiol-sodium salt,
methyl 1-(2,3-dihydro-2,2-dimethyl-1H-inden-1-yl)-1H-imidazole-5-carboxylate,
methyl N-(2,6-dimethylphenyl)-N-(5-isoxazolylcarbonyl)-DL-alaninate,
methyl N-(chloroacetyl)-N-(2,6-dimethylphenyl)-DL-alaninate,
N-(2,6-dimethylphenyl)-2-methoxy-N-(tetrahydro-2-oxo-3-furanyl)-acetamide,
N-(2,6-dimethylphenyl)-2-methoxy-N-(tetrahydro-2-oxo-3-thienyl)-acetamide,
N-(2-chloro-4-nitrophenyl)-4-methyl-3-nitro-benzenesulphonamide,
N-(4-cyclohexylphenyl)-1,4,5,6-tetrahydro-2-pyrimidinamine,
N-(4-hexylphenyl)-1,4,5,6-tetrahydro-2-pyrimidinamine,
N-(5-chloro-2-methylphenyl)-2-methoxy-N-(2-oxo-3-oxazolidinyl)-acetamide,
N-(6-methoxy-3-pyridinyl)-cyclopropanecarboxamide,
N-[2,2,2-trichloro-1-[(chloroacetyl)-amino]-ethyl]-benzamide,
N-[3-chloro-4,5-bis(2-propynyloxy)-phenyl]-N′-methoxy-methanimidamide,
N-formyl-N-hydroxy-DL-alanine-sodium salt,
O,O-diethyl[2-dipropylamino)-2-oxoethyl]-ethylphosphoramidothioate,
O-methyl S-phenyl phenylpropylphosphoramidothioate,
S-methyl 1,2,3-benzothiadiazole-7-carbothioate,
spiro[2H]-1-benzopyran-2,1′(3′H)-isobenzofuran-3′-one,
4-[3,4-dimethoxyphenyl)-3-(4-fluorophenyl)-acryloyl]-morpholine
   Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.
   Insecticides/acaricides/nematicides:
abamectin, acephate, acetamiprid, acrinathrin, alanycarb, aldicarb, aldoxycarb, alpha-cypermethrin, alphamethrin, amitraz, avermectin, AZ-60541, azadirachtin, azamethiphos, azinphos A, azinpbos M, azocyclotin,
Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, betacyfluthrin, bifenazate, bifenthrin, bioethanomethrin, biopermethrin, bistrifluron, BPMC, bromophos A, bufencarb, buprofezin, butathiofos, butocarboxim, butylpyridaben,
cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazine,
deltamethrin, demeton M, demeton S, demeton-S-methyl, diafenthiuron, diazinon, dichlorvos, dicofol, diflubenzuron, dimethoat, dimethylvinphos, diofenolan, disulfoton, docusat-sodium, dofenapyn,
eflusilanate, emamectin, empenthrin, endosulfan, Entomopfthora spp., esfenvalerate, ethiofencarb, ethion, ethoprophos, etofenprox, etoxazole, etrimfos,
fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fubfenprox, furathiocarb,
granulosis viruses,
halofenozide, HCH, heptenophos, hexaflumuron, hexythiazox, hydroprene,
imidacloprid, indoxacarb, isazofos, isofenphos, isoxathion, ivermectin, nuclear polyhedrosis viruses,
lambda-cyhalothrin, lufenuron,
malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,
naled, nitenpyram, nithiazine, novaluron,
omethoate, oxamyl, oxydemethon M,
Paecilomyces fumosoroseus, parathion A, parathion M, permethrin, phenthoat, phorat, phosalone, phosmet, phosphamidon, phoxim, pirimicarb, pirimiphos A, pirimiphos M, profenofos, promecarb, propargite, propoxur, prothiofos, prothoat, pymetrozine, pyraclofos, pyresmethrin, pyrethrum, pyridaben, pyridathion, pyrimidifen, pyriproxyfen,
quinalphos,
ribavirin,
salithion, sebufos, silafluofen, spinosad, spirodiclofen, sulfotep, sulprofos,
tau-fluvalinate, tebufenozide, tebufenpyrad, tebupirimiphos, teflubenzuron, tefluthrin, temephos, temivinphos, terbufos, tetrachlorvinphos, tetradifon, thetacypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,
vamidothion, vaniliprole, Verticillium lecanii,
YI 5302,
zeta-cypermethrin, zolaprofos,
(1R-cis)-[5-(phenylmethyl)-3-furanyl]-methyl 3-[(dihydro-2-oxo-3(2H)-furanylidene)-methyl]-2,2-dimethylcyclopropanecarboxylate
(3-phenoxyphenyl)-methyl 2,2,3,3-tetramethylcyclopropanecarboxylate
1-[(2-chloro-5-thiazolyl)methyl]tetrahydro-3,5-dimethyl-N-nitro-1,3,5-triazine-2(1H)-imine
2-(2-chloro-6-fluorophenyl)-4-[4-(1,1-dimethylethyl)phenyl]-4,5-dihydro-oxazole
2-(acetlyoxy)-3-dodecyl-1,4-naphthalenedione
2-chloro-N-[[[4-(1-phenylethoxy)-phenyl]-amino]-carbonyl]-benzamide
2-chloro-N-[[[4-(2,2-dichloro-1,1-difluoroethoxy)-phenyl]-amino]-carbonyl]-benzamide
3-methylphenyl propylcarbamate
4-[4-(4-ethoxyphenyl)-4-methylpentyl]-1-fluoro-2-phenoxy-benzene
4-chloro-2-(1,1-dimethylethyl)-5-[[2-(2,6-dimethyl-4-phenoxyphenoxy)ethyl]thio]-3(2H)-pyridazinone
4-chloro-2-(2-chloro-2-methylpropyl)-5-[(6-iodo-3-pyridinyl)methoxy]-3(2H)-pyridazinone
4-chloro-5-[(6-chloro-3-pyridinyl)methoxy]-2-(3,4-dichlorophenyl)-3(2H)-pyridazinone
Bacillus thuringiensis strain EG-2348
2-benzoyl-1-(1,1-dimethylethyl)-hydrazinobenzoic acid
2,2-dimethyl-3-(2,4-dichlorophenyl)-2-oxo-1-oxaspiro[4.5]dec-3-en-4-yl butanoate
[3-[(6-chloro-3-pyridinyl)methyl]-2-thiazolidinylidene]-cyanamide
dihydro-2-(nitromethylene)-2H-1,3-thiazine-3(4H)-carboxaldehyde
ethyl [2-[[1,6-dihydro-6-oxo-1-(phenylmethyl)-4-pyridazinyl]oxy]ethyl]-carbamate
N-(3,4,4-trifluoro-1-oxo-3-butenyl)-glycine
N-(4-chlorophenyl)-3-[4-(difluoromethoxy)phenyl]-4,5-dihydro-4-phenyl-1H-pyrazole-1-carboxamide
N-[(2-chloro-5-thiazolyl)methyl]-N′-methyl-N″-nitro-guanidine
N-methyl-N′-(1-methyl-2-propenyl)-1,2-hydrazinedicarbothioamide
N-methyl-N′-2-propenyl-1,2-hydrazinedicarbothioamide
O,O-diethyl [2-(dipropylamino)-2-oxoethyl]-ethylphosphoramidothioate
N-cyanomethyl-4-trifluoromethyl-nicotinamide
3,5-dichloro-1-(3,3-dichloro-2-propenyloxy)-4-[3-(5-trifluoromethylpyridin-2-yloxy)-propoxy]-benzene

A mixture with other known active compounds, such as herbicides, or with fertilizers and growth regulators is also possible.

In addition, the compounds of the formula (I) according to the invention also have very good antimycotic activity. They have a very broad antimycotic activity spectrum, in particular against dermatophytes and yeasts, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

The preparation and the use of the compounds according to the invention is illustrated by the examples below. However, the invention is not limited to the examples.

### PREPARATION EXAMPLES

### Example 1

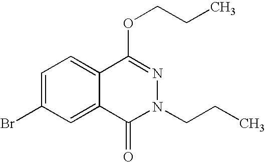 Process a)

At 10° C. and under argon, 12.8 g (0.23 mol) of powdered potassium hydroxide are added to a solution of 25 g (0.1 mol) of 6-bromo-2,3-dihydro-1,4-phthalazinedione in 500 ml of dimethylsulphoxide. At this temperature, 19.3 g (0.23 mol) of iodopropane are then added dropwise, and the mixture is then stirred without further cooling at room temperature overnight. The reaction mixture is poured into 2.5 l of water and extracted three times with in each case 400 ml of ethyl acetate. The combined organic phases are washed twice with in each case 400 ml of water, dried over sodium sulphate and concentrated under reduced pressure. The residue is chromatographed on silica gel using petroleum ether/methyl t-butyl ether (first in a ratio of 40:1, finally 20:1). This gives 14.2 g (42.1% of theory) of 7-bromo-4-propoxy-2-propyl-1(2H)-phthalazinone.

HPLC: logp=4.76

### Example 2

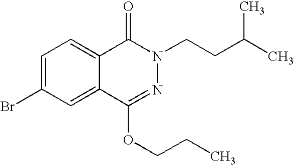 Process b)

0.14 g (1.12 mmol) of powdered potassium hydroxide is added to a solution of 0.5 g (1.77 mmol) of 6-bromo-4-propoxy-1-(2H)-phthalazinone in 10 ml of dimethylsulphoxide. 0.385 g (1.94 mmol) of 1-iodo-3-methylbutane is then added dropwise, and the mixture is then stirred at room temperature overnight. The reaction mixture is poured into 50 ml of water and extracted three times with in each case 50 ml of ethyl acetate. The combined organic phases are washed twice with in each case 50 ml of water, dried over sodium sulphate and concentrated under reduced pressure. The residue is chromatographed on silica gel using petroleum ether/methyl t-butyl ether (20:1). This gives 14.2 g (42.1% of theory) of 6-bromo-2-isopentyl-4-propoxy-1 (2H)-phthalazinone.

HPLC: logp=5.91

### Example 3

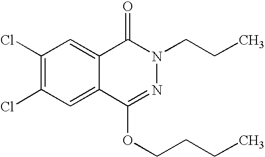 Process c)

0.043 g (0.77-mmol) of powdered potassium hydroxide is added to a solution of 0.2 g (0.73 mmol) of 6,7-dichloro-4hydroxy-2-propyl-1(2H)-phthalazinone in 5 ml of dimethylsulphoxide. 0.27 g (1.46 mmol) of iodobutane is then added dropwise, and the mixture is then stirred at 50° C. for 6 hours. The reaction mixture is poured into 50 ml of water and extracted twice with in each case 50 ml of ethyl acetate. The combined organic phases are washed twice with in each case 50 ml of water, dried over sodium sulphate and concentrated under reduced pressure. The residue is chromatographed on silica gel using cyclohexane/ethyl acetate (3:1). This gives 0.2 g (83% of theory) of 4-butoxy-6,7-dichloro-2-propyl-1 (2H)-phthalazinone.

The compounds of the formula (I) listed in Table 1 can also be obtained analogously to Examples 1 to 3 and in accordance with the general descriptions of processes a), b) and c).

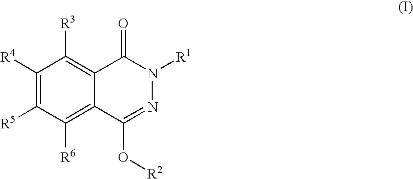

TABLE 1

| Ex. No. | R¹ | R² | R³ | R⁴ | R⁵ | R⁶ | log P* | m.p.(° C.) |
|---|---|---|---|---|---|---|---|---|
| 1 | n-propyl | n-propyl | —H | —Br | —H | H | 4.76 | |
| 2 | i-pentyl | n-propyl | —H | —Br | —H | —H | 5.91 | |
| 3 | n-propyl | n-butyl | —H | —Cl | —Cl | —H | xx | |
| 4 | n-propyl | n-propyl | —H | —H | —Br | —H | 4.8 | |
| 5 | n-butyl | n-propyl | —H | —Br | —H | —H | 5.32 | |
| 6 | allyl | n-propyl | —H | —Br | —H | —H | 4.32 | |
| 7 | propargyl | n-propyl | —H | —Br | —H | —H | 3.82 | |
| 8 | i-butyl | n-propyl | —H | —H | —Br | —H | 5.33 | |
| 9 | 2-butyl | 2-butyl | —H | —Br | —H | —H | 5.52 | |
| 10 | n-propyl | n-propyl | —H | —Cl | —Cl | —H | 5.51 | |
| 11 | n-butyl | n-butyl | —H | —Cl | —Cl | —H | 6.71 | |
| 12 | 2-butyl | 2-butyl | —H | —Cl | —Cl | —H | 3.62 | |
| 13 | i-butyl | i-butyl | —H | —Cl | —Cl | —H | 6.46 | |
| 14 | n-propyl | —CH2— | —H | —Cl | —Cl | —H | | 80 |
| | | C(CH3)3 | | | | | | |
| 15 | n-propyl | n-propyl | —H | —F | —O-n- | —H | 4.82 | |
| | | | | | propyl | | | |
| 16 | n-propyl | n-propyl | —H | —O-n- | —F | —H | 5.26 | |
| | | | | propyl | | | | |
| 17 | n-propyl | n-butyl | —H | —Br | —H | —H | | |
| 18 | n-propyl | n-butyl | —H | —H | —Br | —H | | 72 |
| 19 | n-propyl | -i-propyl | —H | —Cl | —Cl | —H | | 98 |
| 20 | n-propyl | cyclopentyl | —H | —Cl | —Cl | —H | | 83 |
| 21 | n-propyl | cyclohexyl- | —H | —Cl | —Cl | —H | | 93 |
| | | methyl | | | | | | |
| 22 | i-butyl | n-propyl | —H | —Cl | —Cl | —H | | 75 |
| 23 | i-butyl | n-butyl | —H | —Cl | —Cl | —H | | 61 |
| 24 | i-butyl | -i-propyl | —H | —Cl | —Cl | —H | | 79 |
| 25 | i-butyl | cyclopropyl- | —H | —Cl | —Cl | —H | | 75 |
| | | methyl | | | | | | |
| 26 | i-butyl | cyclohexyl- | —H | —Cl | —Cl | —H | | 69 |
| | | methyl | | | | | | |
| 27 | 2- | n-propyl | —H | —Cl | —Cl | —H | | 92 |
| | methoxyethyl | | | | | | | |
| 28 | 2- | n-butyl | —H | —Cl | —Cl | —H | | 89 |
| | methoxyethyl | | | | | | | |
| 29 | 2- | -i-propyl | —H | —Cl | —Cl | —H | | 89 |
| | methoxyethyl | | | | | | | |
| 30 | 2- | i-butyl | —H | —Cl | —Cl | —H | | 73 |
| | methoxyethyl | | | | | | | |
| 31 | 2- | cyclopropyl- | —H | —Cl | —Cl | —H | | 105 |
| | methoxyethyl | methyl | | | | | | |
| 32 | 2- | cyclohexyl- | —H | —Cl | —Cl | —H | | |
| | methoxyethyl | methyl | | | | | | |
| 33 | n-propyl | n-propyl | —H | I | —H | —H | | 99 |
| 34 | n-propyl | n-propyl | —H | —Br | —Br | —H | | 95 |
| 35 | n-propyl | n-propyl | —H | —H | I | —H | | 60 |
| 36 | n-propyl | n-butyl | —H | —Br | —Br | —H | | 113 |
| 37 | n-butyl | -i-propyl | —H | —Cl | —Cl | —H | | |
| 38 | n-butyl | i-butyl | —H | —Cl | —Cl | —H | | |
| 39 | n-butyl | cyclopropyl- | —H | —Cl | —Cl | —H | | 59 |
| | | methyl | | | | | | |
| 40 | n-butyl | cyclopentyl | —H | —Cl | —Cl | —H | | |
| 41 | n-butyl | cyclohexyl- | —H | —Cl | —Cl | —H | | 87 |
| | | methyl | | | | | | |
| 42 | n-propyl | 2-methoxy- | —H | —Cl | —Cl | —H | | 100 |
| | | ethyl | | | | | | |
| 43 | n-propyl | cyclopentyl | —H | —Br | —Br | —H | | 121 |
| 44 | n-propyl | cyclopropyl- | —H | —Br | —Br | —H | | 109 |
| | | methyl | | | | | | |
| 45 | n-propyl | i-butyl | —H | —Br | —Br | —H | | 93 |
| 46 | n-propyl | -i-propyl | —H | —Br | —Br | —H | | 128 |
| 47 | n-propyl | i-butyl | —H | I | —H | —H | | 102 |
| 48 | n-propyl | i-butyl | —H | —H | I | —H | | 88 |
| 49 | n-propyl | cyclohexyl- | —H | I | —H | —H | | 85 |
| | | methyl | | | | | | |
| 50 | n-propyl | cyclohexyl- | —H | —H | I | —H | | 90 |
| | | methyl | | | | | | |
| 51 | n-propyl | -i-propyl | —H | I | —H | —H | | 84 |
| 52 | n-propyl | -i-propyl | —H | —H | I | —H | | 79 |
| 53 | n-propyl | cyclohexyl- | —H | —Br | —Br | —H | | 142 |
| | | ethyl | | | | | | |

*The logP values were determined in accordance with EEC directive 79/831 Annex V.A8 by HPLC (gradient method, acetonitrile/0.1% aqueous phosphoric acid).
**The following compounds were characterized by NMR spectroscopy:
Ex. No. (3)
¹H-NMR (400 MHz, DMSO): δ = 0.89(t, 3H, —CH₃), 3.95(t, 2H, —CH₂—), 8.11(s, 1H, aryl-H) ppm.
Ex. No. (17)
¹H-NMR (400 MHz, DMSO): δ = 0.88(t, 3H, —CH₃), 3.98(t, 2H, —CH₂—), 7.89(d, 1H, aryl-H) ppm.
Ex. No. (32)
¹H-NMR (400 MHz, DMSO): δ = 3.23(s, 3H, —OCH₃), 4.07(d, 2H, —OCH₂—), 8.10(s, 1H, aryl-H) ppm.
Ex. No. (37)
¹H-NMR (400 MHz, DMSO): δ = 0.90(t, 3H, —CH₃), 5.11(m, 1H, —OCH—), 8.07(s, 1H, aryl-H) ppm.
Ex. No. (38)
¹H-NMR (400 MHz, DMSO): δ = 0.90(t, 3H, —CH₃), 4.02(d, 2H, —OCH₂—), 8.09(s, 1H, aryl-H) ppm.
Ex. No. (40)
¹H-NMR (700 MHz, DMSO): δ = 0.90(t, 3H, —CH₃), 5.23(m, 1H, —OCH—), 8.05(s, 1H, aryl-H) ppm.

Preparation of the intermediates of the formula (IV)

### Example (IV-1)

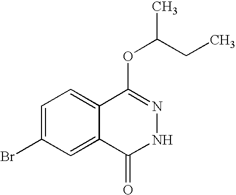 Process d)

1.73 g (26.1 mmol) of powdered potassium hydroxide (purity about 85%) are added to a solution of 6 g (24.9 mmol) of 6-bromo-2,3-dihydro-1,4-phthalazinedione in 90 ml of dimethylsulphoxide. 3.41 g (24.9 mmol) of 2-bromobutane are then added dropwise, and the mixture is then stirred at room temperature overnight. Another 0.49 g (7.47 mmol) of potassium hydroxide and 1.02 g (7.47 mmol) of 2-bromobutane are then added, and stirring is continued for another 24 hours. The reaction mixture is put into 400 ml of water and extracted three times with in each case 120 ml of ethyl acetate. The combined organic phases are washed twice with in each case 150 ml of water, dried over sodium sulphate and concentrated under reduced pressure. The residue is chromatographed repeatedly on silica gel using petroleum ether/methyl t-butyl ether (40:1 to 20:1). This gives 0.76 g (10.3% of theory) of 7-bromo-4-sec-butoxy-1 (2H)-phthalazinone.

HPLC: logP=3.01

The compounds of the formula (IV) listed in Table 2 can be obtained analogously to Example (IV-1) and in accordance with the general description of process d).

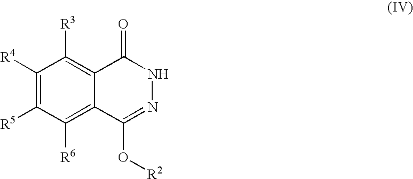

TABLE 2

| Ex. No. | R² | R³ | R⁴ | R⁵ | R⁶ | logP* |
|---|---|---|---|---|---|---|
| (IV-1) | 2-butyl | —H | —Br | —H | —H | 3.01 |
| (IV-2) | n-propyl | —H | —Br | —H | —H | 2.66 |
| (IV-3) | n-propyl | —H | —H | —Br | —H | 2.71 |
| (IV-4) | 2-butyl | —H | —H | —Br | —H | 3.12 |
| (IV-5) | 2-butyl | —H | —Cl | —Cl | —H | 3.62 |
| (IV-6) | i-butyl | —H | —Cl | —Cl | —H | 3.64 |

*The logP values were determined in accordance with EEC directive 79/831 Annex V.A8 by HPLC (gradient method, acetonitrile/0.1% aqueous phosphoric acid).

Preparation of the intermediates of the formula (V)

### Example (V-1)

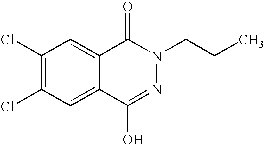 Process e)

A mixture of 1.13 g (5.22 mmol) of 5,6-dichloro-2-benzofuran-1,3-dione, 0.75 g (6.78 mmol) of n-propylhydrazine and 0.54 g of sodium acetate in 10 ml of glacial acetic acid is heated under reflux for 2 hours. After cooling, 100 ml of water are added to the reaction mixture and the resulting precipitate is filtered off, washed with about 30 ml of water and dried. This gives 1.1 g (77% of theory) of 6,7-dichloro-4-hydroxy-2-propyl-1 (2H)-phthalazinone of melting point 223° C.

The compounds of the formula (V) listed in Table 3 can be obtained analogously to Example (V-1) and in accordance with the general description of process e).

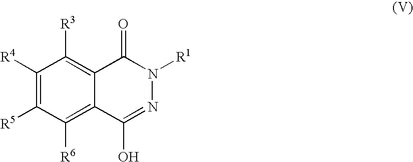

TABLE 3

| | | | | | | | m.p. |
|---|---|---|---|---|---|---|---|
| Ex. No. | R¹ | R² | R³ | R⁴ | R⁵ | R⁶ | (° C.): |
| (V-1) | n-propyl | —H | —H | —Cl | —Cl | —H | 223 |
| (V-2) | n-propyl | —H | —H | —Br | —H | —H | 162 |
| (V-3) | isobutyl | —H | —H | —Cl | —Cl | —H | 217 |
| (V-4) | 2-methoxyethyl | —H | —H | —Cl | —Cl | —H | 223 |
| (V-5) | allyl | —H | —H | —Cl | —Cl | —H | 207 |
| (V-6) | n-propyl | —H | —H | I | —H | —H | 176 |
| (V-7) | n-propyl | —H | —H | —Br | —Br | —H | xx |
| (V-8) | n-butyl | —H | —H | —Cl | —Cl | —H | xx |

xx the following compounds were characterized by NMR:

Ex. No. (V-7)

¹H-NMR (400 MHz, DMSO): δ=0.88 (t, 3H, —CH₃), 3.90 (t, 2H, —CH₂—), 8.20 (s, 1H, aryl-H), 12.0 (s, 1H, —OH) ppm.

Ex. No. (V-8)

¹H-NMR (400 MHz, DMSO): δ=0.90 (t, 3H, —CH₃), 3.95 (t, 2H, —CH₂—), 8.09 (s, 1H, aryl-H), 12.0 (s, 1H, —OH) ppm.

### Example A

Erysiphe test (wheat)/protective

| | |
|---|---|
| Solvent: | 25 parts by weight of N,N-dimethylacetamide |
| Emulsifier: | 0.6 part by weight of alkylaryl polyglycol ether |

To produce a suitable preparation of active compound, I part by weight of active compound is mixed with the stated amounts of solvent and emulsifier, and the concentrate is diluted with water to the desired concentration.

To test for protective activity, young plants are sprayed with the preparation of active compound at the stated application rate.

After the spray coating has dried on, the plants are dusted with spores of Erysiphe graminis fsp. tritici.

The plants are placed in a greenhouse at a temperature of about 20° C. and a relative atmospheric humidity of about 80% to promote the development of mildew pustules.

Evaluation is carried out 7 days after the inoculation. 0% means an efficacy which corresponds to that of the control, whereas an efficacy of 100% means that no infection is observed.

In this test, the compounds of Examples 4, 10 and 11 show, at an application rate of 500 g/ha, an efficacy of 100%.

### Example B

Sphaerotheca test (cucumber)/protective

| | |
|---|---|
| Solvent: | 24.5 parts by weight of acetone |
| | 24.5 parts by weight of dimethylacetamide |
| Emulsifier: | 1.0 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Sphaerotheca fuliginea. The plants are then placed in a greenhouse at about 23° C. and a relative atmospheric humidity of about 70%.

Evaluation is carried out 7 days after the inoculation. 0% means an efficacy which corresponds to that of the control, whereas an efficacy of 100% means that no infection is observed.

In this test, the compounds according to the invention of Examples (5), (6) and (7) show, at an application rate of 100 g/ha, an efficacy of 95% or more.

### Example C

Erysiphe test (cucumber)/protective

| | |
|---|---|
| Solvent: | 25 parts by weight of N,N-dimethylacetamide |
| Emulsifier: | 0.6 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the preparation of active

compound at the stated application rate.

After the spray coating has dried on, the plants are dusted with spores of Erysiphe graminis f.sp. hordei.

The plants are placed in a greenhouse at a temperature of about 20° C. and a relative atmospheric humidity of about 80% to promote the development of mildew pustules.

Evaluation is carried out 7 days after the inoculation. 0% means an efficacy which corresponds to that of the control, whereas an efficacy of 100% means that no infection is observed.

In this test, the compounds of Examples 5, 6 and 11 show, at an application rate of 500 g/ha, an efficacy of 100%.

1-14. (canceled)

15. A compound of formula (I), 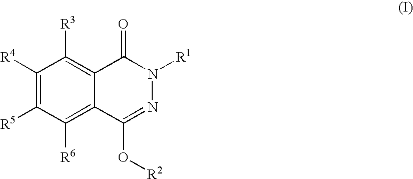
in which
R¹ and R² are identical or different and independently of one another represent alkyl having 2 to 12 carbon atoms, alkenyl, alkynyl, cycloalkyl, cycloalkylalkyl, or alkoxyalkyl, and
R³, R⁴, R⁵, and R⁶ are identical or different and independently of one another represent hydrogen, halogen, cyano, nitro, alkyl, alkoxy, alkylthio, alkylsulphinyl, alkylsulphonyl, alkenyl, alkenyloxy, haloalkyl, haloalkoxy, haloalkylthio, haloalkylsulphinyl, haloalkylsulphonyl, haloalkenyl or haloalkenyloxy, hydroximinoalkyl, alkoximinoalkyl, or cycloalkyl, with the proviso that at least one of the radicals R³, R⁴, R⁵, and R⁶ is not hydrogen.

16. A compound of formula (I) according to claim 15 in which
R¹ and R² are identical or different and independently of one another represent alkyl, alkenyl, or alkynyl having in each case 2 to 12 carbon atoms; cycloalkyl having 3 to 8 carbon atoms; cycloalkylalkyl having 3 to 8 carbon atoms in the cycloalkyl moiety and 1 to 6 carbon atoms in the alkyl moiety; or alkoxyethyl, alkoxypropyl, or alkoxybutyl having in each case 1 to 6 carbon atoms in the alkoxy moiety, and
R³, R⁴, R⁵, and R⁶ are identical or different and independently of one another represent hydrogen, halogen, cyano, or nitro; straight-chain or branched alkyl, alkoxy, alkylthio, alkylsulphinyl, or alkylsulphonyl having in each case 1 to 6 carbon atoms; straight-chain or branched alkenyl or alkenyloxy having in each case 2 to 6 carbon atoms; straight-chain or branched haloalkyl, haloalkoxy, haloalkylthio, haloalkylsulphinyl or haloalkylsulphonyl having in each case 1 to 6 carbon atoms and 1 to 13 identical or different halogen atoms; straight-chain or branched haloalkenyl or haloalkenyloxy having in each case 2 to 6 carbon atoms and 1 to 11 identical or different halogen atoms; hydroxyiminoalkyl having 1 to 6 carbon atoms; alkoxyiminoalkyl having 2 to 6 carbon atoms; or cycloalkyl having 3 to 6 carbon atoms,
where at least one of the radicals R³, R⁴, R⁵, or R⁶ is not hydrogen.

17. A compound of formula (I) according to claim 15 in which
R¹ and R² are identical or different and independently of one another represent straight-chain or branched ethyl, propyl, butyl, pentyl, hexyl, heptyl, octyl, nonyl, decyl, undecyl, or dodecyl; ethenyl, propenyl, butenyl, pentenyl, hexenyl, heptenyl, octenyl, nonenyl, decenyl, undecenyl, or dodecenyl; ethynyl, propynyl, butynyl, pentynyl, hexynyl, heptynyl, octynyl, nonynyl, decynyl, undecynyl, or dodecynyl; cyclopropyl, cyclobutyl, cyclopentyl, or cyclohexyl; cyclopropylmethyl, cyclobutylmethyl, cyclopentylmethyl, cyclohexylmethyl, cyclopropylethyl, cyclobutylethyl, cyclopentylethyl, or cyclohexylethyl; or methoxyethyl, ethoxyethyl, methoxypropyl, or methoxybutyl, each of which may be attached in any position, and
R³, R⁴, R⁵, and R⁶ are identical or different and independently of one another represent hydrogen, fluorine, chlorine, bromine, iodine, cyano, nitro, methyl, ethyl, n- or i-propyl, n-, i-, s-, or t-butyl, methoxy, ethoxy, n- or i-propoxy, methylthio, ethylthio, n- or i-propylthio, methylsulphinyl, ethylsulphinyl, methylsulphonyl, ethylsulphonyl, trifluoromethyl, difluorochloromethyl, fluorodichloromethyl, trifluoroethyl, pentafluoroethyl, difluoromethoxy, trifluoromethoxy, difluorochloromethoxy, trifluoroethoxy, difluoromethylthio, difluorochloromethylthio, trifluoromethylthio, trifluoromethylsulphinyl, trifluoromethylsulphonyl, methylsulphonyloxy, ethylsulphonyloxy, hydroximinomethyl, hydroximinoethyl, methoximinomethyl, ethoximinomethyl, methoximinoethyl, ethoximinoethyl, cyclopropyl, cyclobutyl, cyclopentyl, or cyclohexyl,
where at least one of the radicals R³, R⁴, R⁵, or R⁶ is not hydrogen.

18. A compound of formula (I) according to claim 15 in which
R¹ represents straight-chain or branched methyl, ethyl, propyl, butyl, ethenyl, propenyl, butenyl, ethynyl, propynyl, butynyl, cyclopropyl, cyclobutyl, cyclopentyl, cyclohexyl, cyclopropylmethyl, cyclobutylmethyl, cyclopentylmethyl, cyclohexylmethyl, or methoxyethyl, each of which may be attached in any position,
R² represents straight-chain or branched methyl, ethyl, propyl, butyl, pentyl, ethenyl, propenyl, butenyl, ethynyl, propynyl, butynyl, cyclopropyl, cyclobutyl, cyclopentyl, cyclohexyl, cyclopropylmethyl, cyclobutylmethyl, cyclopentylmethyl, cyclohexylmethyl, or methoxyethyl, each of which may be attached in any position,
R³ represents hydrogen,
R⁴ represents hydrogen, n-propoxy, fluorine, chlorine, bromine, or iodine,
R⁵ represents hydrogen, n-propoxy, fluorine, chlorine, bromine, or iodine, and
R⁶ represents hydrogen.

19. A process for preparing compounds of formula (I) according to claim 15 comprising
(a) reacting a phthalazinedione of formula (II) 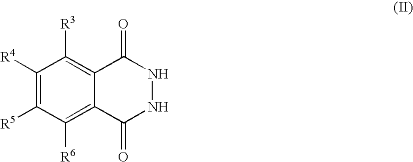
in which R³, R⁴, R⁵, and R⁶ are as defined for formula (I) in claim 15, with an alkyl derivative of formula (III)
R—X (III)
in which
R has the meanings given for R¹ and R² of formula (I) in claim 15, and
X represents a leaving group,
optionally in the presence of an acid acceptor and optionally in the presence of a diluent, or
(b) reacting an alkylphthalazinone of formula (IV) 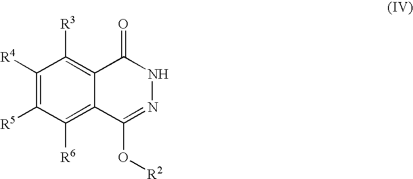
in which R², R³, R⁴, R⁵, and R⁶ are as defined for formula (I) in claim 15, with an alkyl derivative of formula (III)
R—X (III)
in which
R has the meanings given for R¹ of formula (I) in claim 15, and
X represents a leaving group,
optionally in the presence of an acid acceptor and optionally in the presence of a diluent, or
(c) reacting a hydroxyphthalazinone of formula (V) 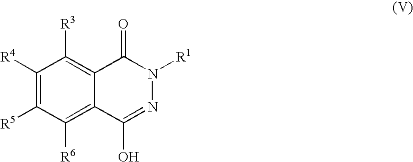
in which
in which R¹, R³, R⁴, R⁵, and R⁶ are as defined for formula (I) in claim 15,
with an alkyl derivative of formula (III),
R—X (III)
in which
R has the meanings given for R² of formula (I) in claim 15, and
X represents a leaving group,
optionally in the presence of an acid acceptor and optionally in the presence of a diluent.

20. A process according to claim 19 wherein the leaving group X is halogen, alkylsulphonyl, or arylsulphonyl.

21. A compound of formula (IV) 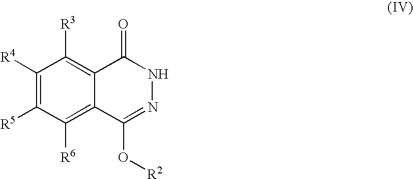
in which
R² represents alkyl having 2 to 12 carbon atoms, alkenyl, alkynyl, cycloalkyl, cycloalkylalkyl, or alkoxyalkyl, and
R³, R⁴, R⁵, and R⁶ are identical or different and independently of one another represent hydrogen, halogen, cyano, nitro, alkyl, alkoxy, alkylthio, alkylsulphinyl, alkylsulphonyl, alkenyl, alkenyloxy, haloalkyl, haloalkoxy, haloalkylthio, haloalkylsulphinyl, haloalkylsulphonyl, haloalkenyl or haloalkenyloxy, hydroximinoalkyl, alkoximinoalkyl, or cycloalkyl,
with the proviso that at least one of the radicals R³, R⁴, R⁵, and R⁶ is not hydrogen.

22. A process for preparing compounds of formula (I) according to claim 21 comprising
(d) reacting a phthalazinedione of formula (II) 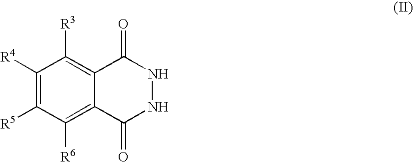
in which R³, R⁴, R⁵, and R⁶ are as defined for formula (IV) of claim 21, with an alkyl derivative of the formula (III)
R—X (III)
in which
R has the meanings given for R² of formula (I) in claim 21, and
X represents a leaving group,
optionally in the presence of an acid acceptor and optionally in the presence of a diluent.

23. A process according to claim 22 wherein the leaving group X is halogen, alkylsulphonyl, or arylsulphonyl.

24. A compound of formula (V) 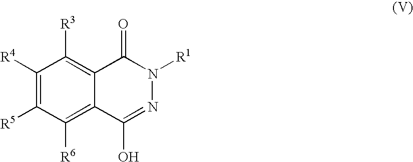
in which
R¹ represents alkyl having 2 to 12 carbon atoms, alkenyl, alkynyl, cycloalkyl, cycloalkylalkyl, or alkoxyalkyl, and
R³, R⁴, R⁵, and R⁶ are identical or different and independently of one another represent hydrogen, halogen, cyano, nitro, alkyl, alkoxy, alkylthio, alkylsulphinyl, alkylsulphonyl, alkenyl, alkenyloxy, haloalkyl, haloalkoxy, haloalkylthio, haloalkylsulphinyl, haloalkylsulphonyl, haloalkenyl or haloalkenyloxy, hydroximinoalkyl, alkoximinoalkyl, or cycloalkyl,
with the proviso that at least one of the radicals R³, R⁴, R⁵, and R⁶ is not hydrogen.

25. A process for preparing compounds of formula (V) according to claim 24 comprising
(e) reacting a phthalic anhydride of formula (VI) 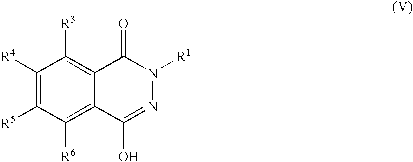
in which R³, R⁴, R⁵, and R⁶ are as defined for formula (V) of claim 24, with a hydrazine derivative of formula (VII)
H₂N—NH—R¹ (VII)
or a salt thereof,
in which R¹ represents alkyl having 2 to 12 carbon atoms, alkenyl, alkynyl, cycloalkyl, cycloalkylalkyl, or alkoxyalkyl,
optionally in the presence of a diluent and optionally in the presence of a salt.

26. A pesticide comprising one or more compounds of formula (I) according to claim 15.

27. A pesticide comprising one or more compounds of formula (IV) according to claim 21.

28. A pesticide comprising one or more compounds of formula (V) according to claim 24.

29. A method for controlling pests comprising allowing an effective amount of a compound of formula (I) according to claim 15 to act on pests and/or their habitat.

30. A method for controlling pests comprising allowing an effective amount of formula (IV) according to claim 21 to act on pests and/or their habitat.

31. A method for controlling pests comprising allowing an effective amount of a compound of formula (V) according to claim 24 to act on pests and/or their habitat.

32. A process for preparing a pesticide comprising mixing a compound of formula (I) according to claim 15 with one or more extenders and/or surfactants.

33. A process for preparing a pesticide comprising mixing a compound of formula (IV) according to claim 21 with one or more extenders and/or surfactants.

34. A process for preparing a pesticide comprising mixing a compound of formula (V) according to claim 24 with one or more extenders and/or surfactants.
